(19) United States
(12) Patent Application Publication
(10) Pub. No.: US 2012/0115209 A1
(43) Pub. Date: May 10, 2012

(54) Method For Culturing Microorganisms, Bioreactor For Implementing Same And Method For Producing Such A Bioreactor

(76) Inventors: Karine Loubiere, Toulouse (FR); Jérémy Pruvost, Saint-Brevin-les-Pins (FR); Jack Legrand, Saint-Nazaire (FR)

(21) Appl. No.: 13/128,033

(22) PCT Filed: Oct. 14, 2009
(86) PCT No.: PCT/FR2009/001205

§ 371 Date: Jun. 3, 2011

(30) Foreign Application Priority Data
Nov. 12, 2008 (FR) 0806296

(51) Int. Cl. C12N 1/12; B32B 37/12; B32B 37/02; C12M 1/00; C12M 1/42

(52) U.S. Cl. 435/257.1; 435/289.1; 435/292.1; 216/33

(57) ABSTRACT

The present invention thus aims to propose a bioreactor which has an improved yield (concentration, productivity), which is capable of culturing microorganisms sensitive to mechanical stresses, and which is energy-saving. The subject matter of the invention is a method for culturing microorganisms in suspension in a culture medium, in which the microorganism suspension is made to flow in a channel which has means for generating mixing by Lagrangian chaos when the microorganism suspension circulates in said channel.

The invention relates to a method for culturing microorganisms, to a bioreactor for implementing that method and to a method for producing such a bioreactor.

The invention concerns the field of culturing microorganisms, and more particularly the culture of photosynthetic microorganisms in bioreactors.

The culture of microorganisms requires an optimized input of nutrients. The culture of photosynthetic microorganisms further requires the most homogeneous input of light possible.

Bioreactors for the culture of photosynthetic microorganisms are called photobioreactors. They are generally based on the same principles as normal bioreactors: the continuous control of the conditions necessary for the optimal growth of the microorganisms, within a volume of culture medium in a reservoir. The main object of current research is to improve the performance thereof (cell concentration and productivity).

The specific feature of photobioreactors lies in the need to provide light energy in addition to the general conditions for culturing. This extra constraint makes these reactors difficult to develop and their geometry difficult to extrapolate. In fact, good utilization of light remains a complex problem in that having strong pigmentation these microorganisms absorb and diffuse the incident light: the light available in the culture medium is thus very rapidly attenuated, especially as the biomass concentration increases. Thus the productivity of the bioreactor is directly controlled by the biological utilization of the incident light.

A first objective of the invention is to improve the conversion yield of light energy into biomass and thus the performance of the photobioreactor (biomass concentration, productivity), in particular when the medium is confined and highly absorbent (large specific areas of illumination).

The state of the art comprises several types of photobioreactor, the geometric configurations whereof are classified on the basis of two categories: a tubular geometry, of the tank, tube or bubble column type, and a planar geometry, known as “flat panel reactor” (FPR).

The feature common to all state of the art photobioreactors is the use of a means for homogenization of the culture medium: a mechanical stirrer within the tank or a bubble circulation for reactors of the bubble column or FPR (air-lift) type.

However, these means for stirring the culture medium induce powerful hydrodynamic stresses which are damaging or even destructive to certain photosynthetic microorganisms known to be fragile.

In addition, the state of the art photobioreactors exhibit low productivity: the concentrations attained very often remain lower than a few grams per liter, and the productivity lower than a few grams per liter per day. This is at present the main barrier to the profitable industrial development of photobioreactors.

At the present time, the only known photobioreactor, the purpose whereof is the intensification of cultures of photosynthetic microorganisms, is the FPR photobioreactor.

Initially introduced by Hu et al in 1996, the FPR is currently the object today of sustained research within the team of Professor R. Wijffels at the University of Wageningen in the Netherlands (“Microalgal photobioreactors: Scale-up and optimization” PhD thesis by Barbosa, 2003). This photobioreactor has a reservoir with a width of 1 to 3 centimeters, a length of 20 centimeters and a height of 60 centimeters. It includes bubble generating injectors distributed over the bottom of the tank. The emission of the bubbles makes it possible to mix the volume of algal suspension.

This FPR photobioreactor obtains attractive performance levels thanks to a large area of exposure relative to the culture volume. In addition, a low level of fouling of the walls is noted, due to the stripping action of the bubbles, which prevents attenuation of the incident light.

However, with the bubble column type systems a high level of gas is necessary to obtain effective stirring of the reaction medium, which can reduce the efficiency of radiative and gas-liquid material transfers (particularly if the microorganism suspension becomes viscous). Further, in this type of configuration, the hydrodynamics of the photosynthetic microorganism suspension are not controlled, which makes it difficult to control the light zone/dark zone cycles to which the photosynthetic microorganisms are sensitive.

A second objective of the present invention is to propose a bioreactor suitable for the culture of microorganisms known to be sensitive to mechanical stresses while at the same time having improved performance (concentration/productivity) and limiting the fouling of the walls of the bioreactor.

In order to remedy the disadvantages of the known solutions, the present invention proposes a method and a bioreactor in which the culture medium is made to flow in a geometric structure generating mixing by Lagrangian chaos.

The mixing by Lagrangian chaos should not be confused with random or turbulent mixing, sometimes wrongly described as “chaos”, under the pretext that the medium is stirred vigorously. Thus, in fluid mechanics, mixing is described as Lagrangian chaos if it satisfies at least one of the following criteria:
sensitivity to the initial conditions (also called the law of divergent trajectories).
In a chaotic system, extremely close initial conditions can develop very differently: it is then said that there is a loss of memory of the initial conditions. For mixing by Lagrangian chaos, the divergence between two initially very close fluid trajectories grows exponentially. Nevertheless, as this system is determinist, the same initial condition will always give the same final state. This is the main difference that exists between mixing by Lagrangian chaos and random (for example turbulent) mixing. In fact, random mixing associates different final states with the same initial condition.
presence of homoclinic or heteroclinic transverse intersections.
In other words, the presence of at least one unstable element in a system certainly complicates its dynamics, but above all makes it more effective in terms of mixing. From a mathematical point of view, the unstable points are typically hyperbolic points (unlike the elliptical points which are obstacles to the mixing). Two local behaviors are associated with hyperbolic points: one stable and the other unstable, corresponding respectively to the physical directions of maximum compression and stretching. The intersection of these two behaviors generates either from points referred to as homoclinic (if emanating from the same hyperbolic point), or from points referred to as heteroclinic (if emanating from two hyperbolic points). These homoclinic and heteroclinic intersections are an indicator of Lagrangian chaos.
horseshoe transformation of a reference volume.
In mixing by Lagrangian chaos, a unit volume of fluid is stretched in one direction, which causes it to contract in a perpendicular direction, then it is turned back on its initial position of origin. Thus, for an initial cube-shaped volume mixing by Lagrangian chaos is characterized by stretching and turning back of the fluid trajectories in a horseshoe shape.

Finally it should be noted that according to the theory of dynamic systems the chaotic movement of particles can only take place when the velocity field is either two-dimensional and time-dependent or three-dimensional whether or not time-dependent.

The invention relates to the use of a channel capable of generating mixing by Lagrangian chaos when fluid circulates within said channel as a bioreactor for the culture of microorganisms. The flow is thus described, in the remainder of this description, as “chaotic”. In a preferred application, the invention uses a channel transparent to radiation for the culture of photosynthetic microorganisms.

The subject matter of the present invention is a method for culturing microorganisms in suspension in a culture medium, in which the microorganism suspension is made to flow in a channel which has means for generating mixing by Lagrangian chaos when the microorganism suspension circulates in said channel.

Through this method, the nutrient supply of cells is optimized thanks to the circulation of fluid within the channel. In addition, owing to its laminar nature along the whole length of the channel, the chaotic flow of the culture medium itself, and not the mixing of a static medium, enables the culture of microorganisms known to be sensitive to mechanical stresses (such as certain dinoflagellates such as Protoceratum reticulum or diatoms such as Skeltonema costatum), while at the same time saving energy.

This method offers an original solution for the culture of microorganisms, making it possible to obtain, thanks to the three-dimensional nature of the chaotic flow (successive turning back/stretching of lines of fluid), a mixing equivalent to that obtained in a turbulent system, but without the high mechanical stresses present in any flow other than laminar.

The advantage due to these properties of mixing by Lagrangian chaos is, in particular when the medium is confined and highly absorbent (high specific surface area of illumination), an improvement in the exposure of the photosynthetic microorganisms to light radiation (better spatial homogeneity and light penetration), thus allowing an increase in the biological performance (cell concentration, productivity) of the culture.

However, the mixing by Lagrangian chaos implemented in the present invention offers a further advantage. In fact, although it does not induce any strong mechanical stress in the culture medium, unlike the bubbles in the FPR, it is noted that the fouling of the channel is markedly limited, that is to say the microorganisms hardly adhere at all to the wall of the channel, even after several tens of days of circulation in the channel. Mixing by Lagrangian chaos therefore makes it possible to reconcile two previously incompatible advantages: effective mixing which respects the cellular integrity of the microorganisms, and limited fouling of the bioreactor.

According to other embodiments:
the channel can comprise a plurality of basic forms connected to one another by connecting arms;
the basic forms can be selected from the group made up of “C”-shaped forms, “V”-shaped forms, “B”-shaped forms, “U”-shaped forms, “3D zigzag”-shaped forms, channels with alternating circular segments, “L”-shaped forms and a mixture thereof;
the method can be adapted for the culture of photosynthetic microorganisms, and the channel can have walls transparent to the light radiation necessary for the growth of the photosynthetic microorganisms to be cultured;
the method can include a step of circulation of the cell suspension in at least one reservoir arranged in series in the channel;
the method can include a step of control of the quantity of gas in the culture medium; and/or
the method can include a step of control of the temperature of the culture medium.

The invention also relates to a bioreactor for the culture of microorganisms in suspension in a culture medium, comprising a channel equipped with an inlet and an outlet for the microorganism suspension and a means for making the microorganism suspension flow in the channel, the channel having means for generating mixing by Lagrangian chaos when the microorganism suspension circulates within it.

According to other embodiments:
the means for generating mixing by Lagrangian chaos can comprise a plurality of basic forms connected to one another by connecting arms;
the basic forms can be selected from the group made up of “C”-shaped forms, “V”-shaped forms, “B”-shaped forms, “U”-shaped forms, “3D zigzag”-shaped forms, channels with alternating circular segments, “L”-shaped forms and a mixture thereof;
the bioreactor can be adapted for the culture of photosynthetic microorganisms, and the channel can have walls transparent to the light radiation necessary for the growth of the photosynthetic microorganisms to be cultured;
the channel can include at least one reservoir arranged in series in the channel;
the channel can include means for control of the quantity of gas in the culture medium; and/or
the bioreactor can further include a heat exchanger to control the temperature of the culture medium.

The invention also relates to a method for producing such a bioreactor, the method comprising the following steps:
etching, in a first plate of substrate, a plurality of basic forms selected from the group made up of “C”-shaped forms, “V”-shaped forms, “B”-shaped forms, “U”-shaped forms, “3D zigzag”-shaped forms, channels with alternating circular segments, “L”-shaped forms and a mixture thereof;
etching, in a first plate of substrate, a channel inlet and outlet;
etching, in a second plate of substrate, a plurality of connecting arms;
deposition of a sealing joint and juxtaposition of the two plates of substrate thus etched in such a way that the connecting arms are arranged in such a manner as to allow watertight fluidic communication between the basic forms and with the channel inlet and outlet.

According to other embodiments:
the production method can further include a step of etching in the first plate at least one reservoir intended to be arranged in series in the channel;
the production method can further include a step of etching in the second plate at least one reservoir intended to be arranged in series in the channel; and/or
the production method can further include a stage of etching in a third plate of substrate a channel for circulation of a heat-transfer fluid, and a stage of juxtaposition of the first and third plates of substrate thus etched in such a way that the channel for circulation of heat-transfer fluid is in a heat exchange relationship with the channel for circulation of the culture medium.

Other characteristics of the invention will be outlined in the following detailed description with reference to the appended figures, which represent respectively;

FIG. 1, a schematic view from above of a practical example of a bioreactor according to the invention;

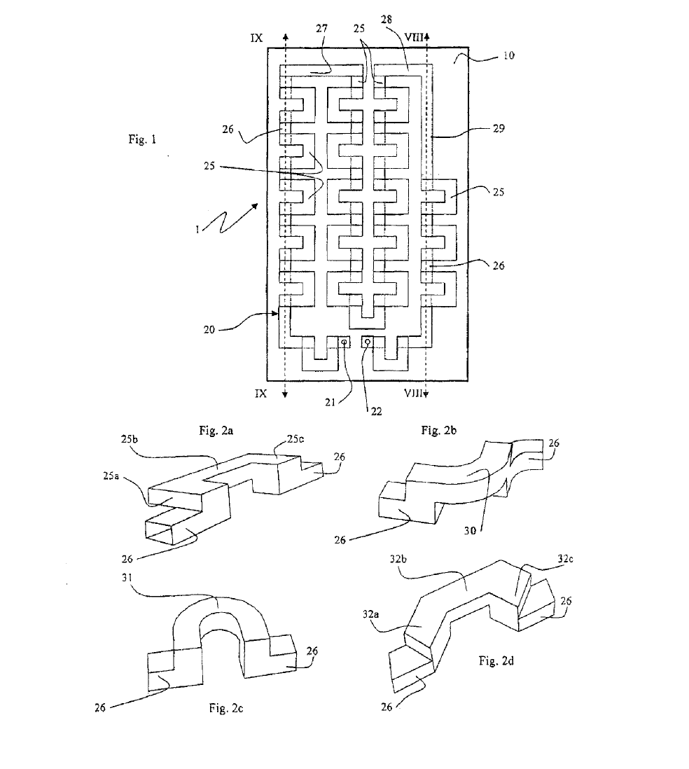

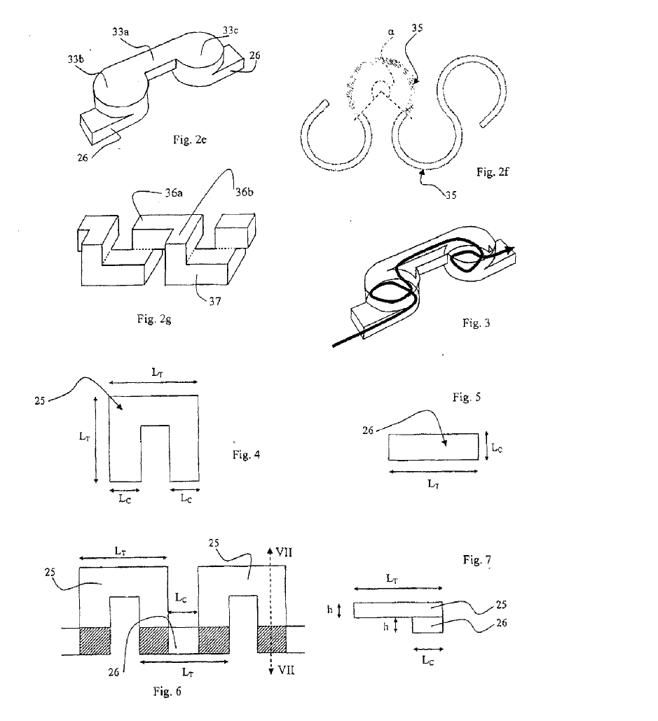

FIGS. 2a to 2g, schematic perspective views of different possible geometries of basic forms and of connecting arms of a channel according to the invention;

FIG. 3, a schematic perspective view illustrating a microorganism trajectory in the chaotic flow of the microorganism suspension in the geometry of FIG. 2e;

FIG. 4, a schematic view from above of a “C”-shaped basic form;

FIG. 5, a schematic view from above of a connecting arm for basic forms;

FIG. 6, a schematic view from above of a portion of a channel according to the invention comprising “C”-shaped basic forms linked by connecting arms;

FIG. 7, a schematic sectional view of the portion of channel in FIG. 6 along the cut line VII-VII; and

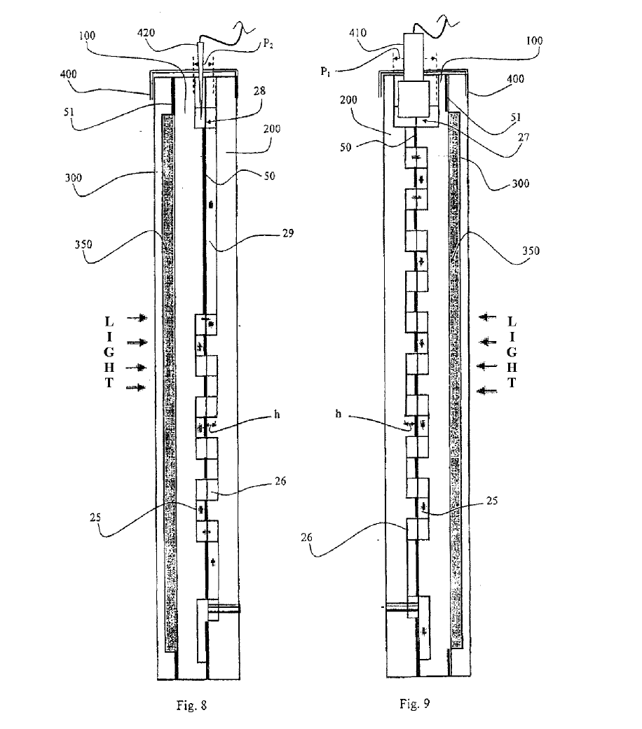

FIGS. 8 and 9, schematic views from the right and left sides of a practical example of the bioreactor of FIG. 1.

A preferred example of application of the invention proposes the use of a channel with a structure capable of generating mixing by Lagrangian chaos when the culture medium flows into said channel as a bioreactor for the culture of microorganisms.

The bioreactor according to the invention on the one hand enables mixing which is effective but generates few mechanical stresses and on the other hand minimal fouling of the walls of the channel thanks to the three-dimensionality of the flow and the velocities near the wall which are several times greater than the average velocity of circulation (calculated by the ratio between the flow rate and the cross section), and all this even in the presence of millimeter culture thicknesses.

In order to intensify the performance (concentrations, productivity), the channel used has a reduced culture thickness (less than one centimeter) and therefore an increased specific area of illumination (greater than 130 m⁻¹) compared to the state of the art photobioreactors, thus making the medium particularly confined and strongly absorbent.

In a preferred geometric form (“C”-shaped forms), the Reynolds number is of the order of 200: it should be optimized in terms of the efficiency of the generated chaotic flow (mixing) and flow velocities sufficient to maintain the photosynthetic microorganisms in suspension (of the order of several centimeters per second). Incidentally, the kinematic viscosity of a 1 g/L algal suspension is close to that of water, but it increases with the cell concentration.

Thus, the channel used has reduced dimensions to promote the exposure of microorganisms to the light, while ensuring, on the one hand, effective circulation of the fluid containing microorganisms in relatively high concentration and on the other hand good profitability of the bioreactor.

In general, the Reynolds number of the flow in a bioreactor according to the invention is between 150 and 250, preferably 200.

The bioreactor 1 illustrated in FIG. 1 includes a support 10 of a channel 20. This channel 20 includes a fluidic inlet 21 and a fluidic outlet 22.

The fluidic inlet 21 is made up of a single aperture constructed in such a manner that the photosynthetic microorganisms in suspension in the culture medium do not undergo mechanical constraints or stress capable of damaging them.

A means (not shown) of making a fluid circulate in the channel is in fluidic communication with the channel via the inlet 21 and outlet 22. By way of example, this means is a peristaltic pump capable of making the suspended microorganisms circulate and recirculate in the channel 20 without damaging them.

The channel 20 makes it possible to generate a chaotic flow when the fluid circulates in said channel.

According to the embodiment illustrated in FIG. 1, channel 20 includes a plurality of basic forms 25 connected to one another by connecting arms 26.

The basic forms are generally made up of a tubular coil causing changes of direction of the circulating fluid.

In the embodiment in FIG. 1, the basic forms 25 are “C”-shaped forms connected by straight connecting arms 26 described in FIG. 5. Each “C”-shaped form is a tubular coil comprising three successive rectilinear sections 25a, 25b, 25c (see FIG. 2a) arranged at right angles. The arrangement of the sections is coplanar.

When the microorganisms cultivated are photosynthetic microorganisms, the channel 20 has walls transparent to the light radiation necessary for the growth of the microorganisms to be cultured.

In the embodiment illustrated, the channel contains two reservoirs 27-28 in fluidic communication with the basic units 25.

These reservoirs make it possible to use standard instruments 410 (see FIG. 9) for monitoring parameters such as the pH and the temperature and instruments for regulating these parameters or instruments for sampling and/or injection 420 (see FIG. 8). By way of example, a standard and economical pH probe has a diameter of about 12 mm and could not be integrated directly into the channel 20.

Alternatively, it is possible to omit these reservoirs if the measurement instruments can be integrated directly into the channel. However, this type of instrumentation gives rise to considerable costs due to its miniaturization.

In order to limit the perturbations caused by these reservoirs on the chaotic flow of the culture medium, it is preferable to group the bulky instruments together in a first reservoir 27 having relatively large dimensions (for example, four times the height of the channel). Likewise, the less bulky instruments are grouped together in a second reservoir 28 of more modest dimensions (for example, twice the height of the channel). By way of example, the second reservoir 28 can serve for placing the instruments for sampling and/or injection of cell suspension.

The bioreactor of FIG. 1 also includes a linear section 29 placed at the outlet of the second reservoir 28. This linear section 29 can contain means for control of the quantity of gas present in the culture medium. It is thus possible to inject air and/or CO₂ in order to optimize the growth conditions of the photosynthetic microorganisms (pH, quantity of carbon available for photosynthesis, removal of the O₂ produced, etc.).

In order to limit the risk of accumulation of bubbles in the channel, it is possible to omit this linear section and/or to inject carbonic acid H₂CO₂ in liquid form in place of the gas (air/CO₂).

Several types of basic forms 25 and connecting arms 26 can be used, provided that they enable mixing by Lagrangian chaos when the fluid circulates within them. The choice of one or the other results from a compromise between:
technological simplicity (production and dismantling of the bioreactor),
the efficiency of the chaotic flow (optimization between the mixing and the improvement of the exposure of the photosynthetic microorganisms),
the minimization of energy consumption,
and the minimization of the fouling of the walls of the channel and respect of the sensitivity to mechanical stresses.

Seven non-limiting examples are illustrated in FIGS. 2a to 2g.

In FIGS. 2a to 2e, the forms are illustrated in fluidic communication with two connecting arms 26, shown in part. The forms 2b to 2e are modifications of the “C”-shaped form developed at the LTN Laboratory of the University of Nantes in France (team of Professor H. Peerhossaini, C. Castelain and B. Auvity) with the aim of maximizing the performance of the fuel cell heat exchangers. The Applicant only had knowledge of this work through the good fortune of geographical proximity to this laboratory. Although the technical fields are very different, the Applicant had the idea of testing these forms in the specific application of bioreactors and in particular photobioreactors. It perceived that the utilization of these forms 2b to 2e in a chaotic advection bioreactor would significantly improve the cell concentration and the productivity while limiting the fouling of the channel and thus favoring the exposure to light.

The basic form illustrated in FIG. 2a is a “C”-shaped form like those which were described for FIG. 1. The basic form illustrated in FIG. 2b is referred to as “3D zigzag”-shaped. It comprises a curved section 30 making an essentially right-angled bend.

The basic form illustrated in FIG. 2c is a form referred to as “U”-shaped. In this embodiment, the fluid effects a curved motion (secondary flow) in a bent section 31, whereas in the C-shaped form the fluid effects a rectilinear motion within each rectilinear section. The U-shaped form generates less pressure loss than the C-shaped form.

The embodiment illustrated in FIG. 2d is referred to as a “V-shaped form”. This modification makes it possible to create a spatially chaotic flow with less pressure loss than the C-shape form owing to angles less than 90° between the rectilinear sections 32a, 32b and 32c.

The embodiment illustrated in FIG. 2e is referred to as “B”-shaped. This basic form comprises a rectilinear section 33a at the ends whereof cylindrical sections 33b and 33c are positioned. Thus, as is shown by FIG. 3, the fluid locally effects a turbulent flow induced by the cylindrical sections within the basic form and, in the set of forms, the fluid undergoes a spatially chaotic flow.

The embodiment of FIG. 2f is referred to as “having alternating circular segments”. Each basic form 35 is made up of a channel portion shaped like an arc of a circle, of square cross section, of radius of curvature equal to five times the diameter of the channel and of length defined by a span α essentially equal to three quarters of a circle. Thus the channel possesses two straight inlet and outlet sections, and a succession of basic forms 35 shaped like arcs of circles oppositely positioned. It is precisely these periodic changes of direction which generate mixing by Lagrangian chaos.

The embodiment of FIG. 2g is referred to as “L”-shaped. Another modification of the “C”-shaped form, this form comprises two straight sections 36a and 36b of different lengths and perpendicular to one another. The connecting arms 37 are identical to the “L”-shaped form.

According to a preferred embodiment, illustrated in FIG. 4, the C-shaped basic form has a total width L_{T} equal to three times the width L_{C} of the channel. By way of example, the width of the channel L_{C} is equal to 10 mm. The total width L_{T} is thus equal to 30 mm. Likewise, in a preferred embodiment, illustrated in FIG. 5, each connecting arm comprises a total width L_{T} equal to three times the width L_{C} of the channel. The combination of these basic forms and connecting arms thus dimensioned is illustrated in FIG. 6.

FIG. 7 is a cross section of the entirety of FIG. 6 along the cut line VII-VII. The basic forms 25 and the connecting arms 26 are of equal height h, preferably, at about half of the width L_{C} of the channel. In other words, the height h is approximately equal to 5 mm for the example mentioned above.

According to a preferred embodiment, the bioreactor according to the invention is implemented in at least two juxtaposed plates of transparent material. This structure is illustrated in FIGS. 8 and 9.

The bioreactor comprises a first and a second plate 100-200 in which the basic forms 25, the connecting arms 26, the fluidic inlet 21, the fluidic outlet 22 and, in the embodiment illustrated, two reservoirs 27-28 of different depths and a straight section 29 have been etched.

A sealing joint 50 is positioned between the two plates 100-200 and these are juxtaposed in such a manner that the connecting arms 26 are positioned so as to allow watertight fluidic communication between the basic forms 25 and with the inlet 21 and the outlet 22 of channel 20.

In the embodiment illustrated, the first reservoir 27 has a depth P₁ approximately equal to four times the height h of the channel. To produce this reservoir, the two plates 100 and 200 are etched to a depth approximately equal to twice the height of the channel.

In the same way, the reservoir 28 has a depth P₂ approximately equal to twice the height h of the channel. To produce this reservoir, the two plates 100 and 200 are etched to a depth approximately equal to the height of the channel.

Optionally, the bioreactor includes a third plate 300 in which there is etched a circuit 350 for circulation of a heat transfer fluid to regulate the temperature of the culture medium by heat exchange.

Preferably, the plate 300 is juxtaposed to the plate 100 bearing the basic forms 25. This improves the heat exchange area between the heat transfer fluid and the culture medium. A sealing joint 51 is positioned between the two plates 100 and 300 to ensure water-tightness.

This heat transfer fluid must be transparent to the radiation useful for the growth of the photosynthetic microorganisms. Preferably, this fluid is water.

Preferably, the bioreactor includes a detachable lid 400 intended to serve as a watertight support for the measurement instruments 410 in the reservoir(s) 27-28. This lid 400 can also include apertures for the passage of needles 420 for sampling or injection of microorganisms.

In the embodiment illustrated, the photobioreactor according to the invention is utilized vertically. This position makes it possible to facilitate the removal towards the upper part of the bioreactor of the bubbles of gas which might be formed in the reaction medium.

Numerous modifications and alternatives can be made without thereby departing from the invention and in particular:
the bioreactor can include basic forms the walls whereof are constructed to favor the removal of the bubbles towards the top of the reservoir when this is used vertically.
the bioreactor can include a combination of basic forms of different geometries generating mixing by Lagrangian chaos when fluid circulates within it.

1. A method for culturing microorganisms in suspension in a culture medium, characterized in that the microorganism suspension is made to flow in a channel which has means for generating mixing by Lagrangian chaos when the microorganism suspension circulates in said channel.

2. The method as claimed in claim 1, in which the channel comprises a plurality of basic forms connected to one another by connecting arms.

3. The method as claimed in claim 2, in which the basic forms are selected from the group made up of “C”-shaped forms, “V”-shaped forms, “B”-shaped forms, “U”-shaped forms, “3D zigzag” forms, channels with alternating circular segments, “L”-shaped forms and a mixture thereof.

4. The method as claimed in claim 1 for the culture of photo-synthetic microorganisms, in which the channel has walls transparent to the light radiation necessary for the growth of the photosynthetic microorganisms to be cultured.

5. The method as claimed in claim 1, including a step of circulation of the cell suspension in at least one reservoir arranged in series in the channel.

6. The method as claimed in claim 1, including a step of control of the quantity of gas in the culture medium.

7. The method as claimed in claim 1, including a step of control of the temperature of the culture medium.

8. A bioreactor for the culture of microorganisms in suspension in a culture medium, wherein the bioreactor comprises a channel equipped with an inlet and an outlet for the microorganism suspension and a means for making the microorganism suspension flow in the channel, the channel having means for generating mixing by Lagrangian chaos when the microorganism suspension circulates in said channel.

9. The bioreactor as claimed in claim 8, in which the means for generating mixing by Lagrangian chaos comprises a plurality of basic forms connected to one another by connecting arms.

10. The bioreactor as claimed in claim 9, in which the basic forms are selected from the group made up of “C”-shaped forms, “V”-shaped forms, “B”-shaped forms, “U”-shaped forms, “3D zigzag” forms, channels with alternating circular segments, “L”-shaped forms and a mixture thereof.

11. The bioreactor as claimed in claim 8 for the culture of photosynthetic microorganisms, in which the channel has walls transparent to the light radiation necessary for the growth of the microorganisms to be cultured.

12. The bioreactor as claimed in claim 8, in which the channel includes at least one reservoir arranged in series in the channel.

13. The bioreactor as claimed in claim 8, including means for controlling the quantity of gas in the culture medium.

14. The bioreactor as claimed in claim 8, further including a heat exchanger for controlling the temperature of the culture medium.

15. A method for the manufacture of a bioreactor as claimed in claim 10, comprising the following steps:
etching, in a first plate of substrate, of a plurality of basic forms selected from the group made up of “C”-shaped forms, “V”-shaped forms, “B”-shaped forms, “U”-shaped forms, “3D zigzag” forms, channels with alternating circular segments, “L”-shaped forms and a mixture thereof;
etching, in a first plate of substrate, of an inlet and an outlet to/from channel;
etching, in a second plate of substrate, of a plurality of connecting arms;
deposition of a sealing joint and juxtaposition of the two plates of substrate thus etched in such a way that the connecting arms are arranged in such a manner as to allow watertight fluidic communication between the basic forms and with the channel inlet and outlet.

16. The production method as claimed in claim 15, further including a step of etching in the first plate at least one reservoir intended to be arranged in series in the channel.

17. The production method as claimed in claim 16, further including a step of etching in the first plate at least one reservoir intended to be arranged in series in the channel.

18. The production method as claimed in claim 17, further including a stage of etching, in a third plate a channel for circulation of a heat-transfer fluid, and a stage of juxtaposition of the first and third plates of substrate thus etched in such a way that the channel for circulation of heat-transfer fluid is in a heat exchange relationship with the channel for circulation of the culture medium.
